(19) United States
(12) Patent Application Publication
(10) Pub. No.: US 2010/0261959 A1
(43) Pub. Date: Oct. 14, 2010

(54) In-vivo observation system and method for driving in-vivo observation system

(75) Inventor: Naohito DOI, Kamiina-gun (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/752,344

(22) Filed: Apr. 1, 2010

(30) Foreign Application Priority Data
Apr. 3, 2009 (JP) 2009-091335

(51) Int. Cl. A61B 1/04

(52) U.S. Cl. 600/109

(57) ABSTRACT

An in-vivo observation system of the present invention is provided with an in-vivo observation apparatus including: a living-body information acquiring section that acquires living-body information; a transmission section that transmits the living-body information to outside the living body; a power source section that supplies driving power to the living-body information acquiring section and the transmission section; a magnetic field detection section that detects an AC magnetic field from outside and outputs a detection result as an electric signal; and a power supply control section that controls the supply state of the driving power that is supplied to the living-body information acquiring section and the transmission section, based on the electric signal; and a power source starter of the in-vivo observation apparatus in which a magnetic field generating section that generates an AC magnetic field and a display section are integrally formed.

This application claims benefit of Japanese Application No. 2009-091335 filed in Japan on Apr. 3, 2009, the contents of which are incorporated by this reference.

### BACKGROUND OF THE INVENTION

1. Field of the Invention

The present invention relates to an in-vivo observation system provided with an in-vivo observation apparatus and a power source starter for the in-vivo observation apparatus, the power source starter being disposed outside the in-vivo observation apparatus, and a method for driving the in-vivo observation system.

2. Description of Related Art

In recent years, micro endoscopes, so-called capsule endoscopes, which include a photographing unit and an illumination optical system, etc. in, for example, a housing of a tablet capsule shape have been developed.

A capsule endoscope is introduced into a body cavity by means of swallowing by an examinee, for example, and picks up images of an affected area, etc. to transmit the images to outside the body.

Receiving transmitted images at outside the body allows an observation and inspection, etc. of the inside of the body cavity to be performed. Therefore, a capsule endoscope has an advantage in that an observation or an inspection, etc. of organs such as a small intestine, which have been difficult to be observed or inspected by a conventional endoscope having an insertion portion, can be performed with relative ease.

Moreover, a method for using a magnetic material is well known as a method for controlling the activation/stopping of a capsule endoscope, specifically for controlling the start and stop of image pickup, and controlling the start and stop of illumination, from outside in a non-contact manner at any timing, and is disclosed in Japanese Patent Application Laid-Open Publication No. 2006-94933.

Japanese Patent Application Laid-Open Publication No. 2006-94933 discloses the configuration of an in-vivo observation system in which a reed switch is used for the switch for turning on/off the power supply to each component in a capsule endoscope from a battery provided in the capsule endoscope.

Using a reed switch for the power supply of a capsule endoscope enables to activate the capsule endoscope by applying a magnetic force in a non-contact manner to the capsule endoscope before it passes through the mouth, by use of a magnetic body provided in an external apparatus, thereby switching the reed switch from Off to On by the magnetic force.

Moreover, it is possible to allow the operator to recognize the power supply state to the capsule endoscope through the lighting and non-lighting of an LED provided in the capsule endoscope.

Further, Japanese Patent Application Laid-Open Publication No. 2006-94933 discloses a configuration which is provided with a magnetic body (permanent magnet) for turning on/off a reed switch of the capsule endoscope, and in which an external apparatus constituting a power source starter is provided with a display section which can display picked up images that are picked up and transmitted by the capsule endoscope; that is, a configuration of a compact external apparatus, in which a display section and a power source starter are integrated, is disclosed, which allows for the recognition of the power supply state and communication state of the capsule endoscope by means of the images displayed on the display section as well.

### SUMMARY OF THE INVENTION

Briefly, an in-vivo observation system of the present invention is provided with an in-vivo observation apparatus including a living-body information acquiring section that acquires living-body information in a living body; a transmission section that wirelessly transmits the living-body information to outside the living body, a power source section that supplies driving power to the living-body information acquiring section and the transmission section, a magnetic field detection section that detects an AC magnetic field from outside and outputs a detection result as an electric signal, and a power supply control section that controls the supply state of the driving power that is supplied from the power source section to the living-body information acquiring section and the transmission section, based on the electric signal; and a power source starter for the in-vivo observation apparatus, which is disposed outside the in-vivo observation apparatus, and in which a magnetic field generating section that generates an AC magnetic field and a display section are integrally formed.

Moreover, a method for driving an in-vivo observation system according to the present invention is a method for driving the in-vivo observation system including: an in-vivo observation apparatus including a living-body information acquiring section that acquires living-body information in a living body, a transmission section that transmits the living-body information to outside the living body, a power source section that supplies driving power to the living-body information acquiring section and the transmission section, a magnetic field detection section that detects an AC magnetic field from outside and outputs a detection result as an electric signal, and a power supply control section that controls the supply state of the driving power that is supplied from the power source section to the living-body information acquiring section and the transmission section, based on the electric signal; and a power source starter for the in-vivo observation apparatus, which is disposed outside the in-vivo observation apparatus, and in which a magnetic field generating section that generates an AC magnetic field and a display section are integrally formed, wherein the power supply state of the in-vivo observation apparatus is switched from On to Off, or Off to On every time when the AC magnetic field is emitted from the magnetic field generating section.

The above and other objects, features and advantages of the invention will become more clearly understood from the following description referring to the accompanying drawings.

### BRIEF DESCRIPTION OF THE DRAWINGS

FIG. 1 shows an outline of the configuration of an in-vivo observation system showing an embodiment of the present invention;

FIG. 2 shows an outline of the configuration of an electric circuit of an AC magnetic field generating apparatus that is included in a power source starter of FIG. 1;

FIG. 3 shows an outline of the configuration of an electric circuit of a capsule endoscope of FIG. 1;

FIG. 4 schematically shows an external appearance of the power source starter of FIG. 1;

FIG. 5 shows a variant in which a primary side coil of a transmission antenna is provided on the top surface of the power source starter;

FIG. 6 shows a variant in which the planar shape of the primary side coil of the transmission antenna of FIG. 4 is formed into a figure of 8;

FIG. 7 is a timing chart to show the activation and stop operations of a capsule endoscope by use of an AC magnetic field; and

FIG. 8 schematically shows a situation in which the activation/stopping of the capsule endoscope of FIG. 1, which has been introduced into a body after being passed through the mouth, is performed through the generation of an AC magnetic field by a power source starter, which includes a display section on which an observed image picked up by the capsule endoscope is displayed.

### DETAILED DESCRIPTION OF PREFERRED EMBODIMENTS

Hereafter, embodiments of the present invention will be described with reference to the drawings. It is noted that the drawings are schematic illustration, in which the relationship between the thickness and width of each component and the proportion of the thicknesses of respective components, etc. are different from those of reality; and of course include portions in which dimensional relationship and proportion are different from one another.

Moreover, in the following embodiment, an in-vivo observation apparatus will be described taking a capsule endoscope as an example.

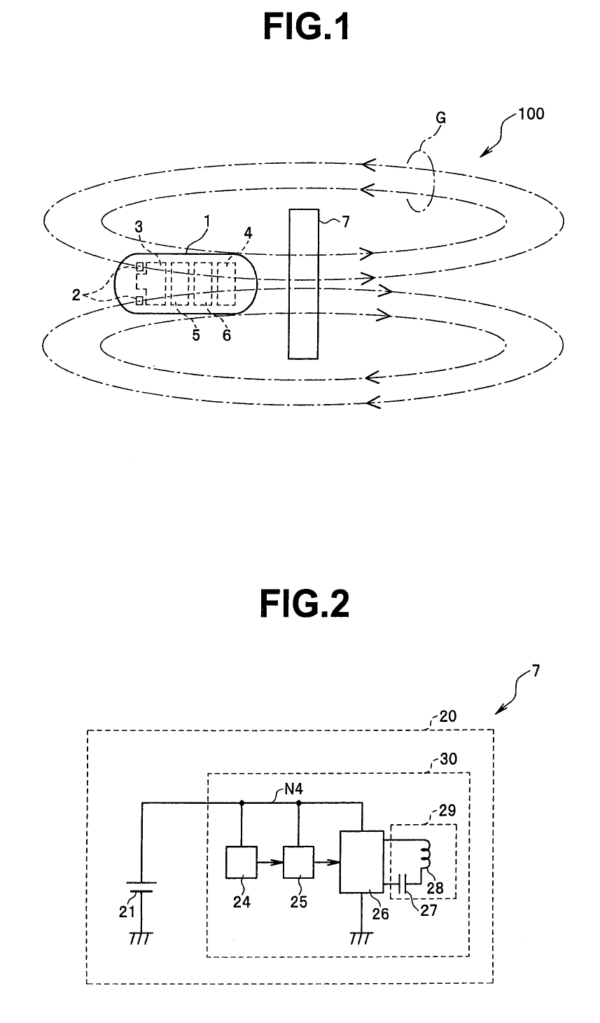

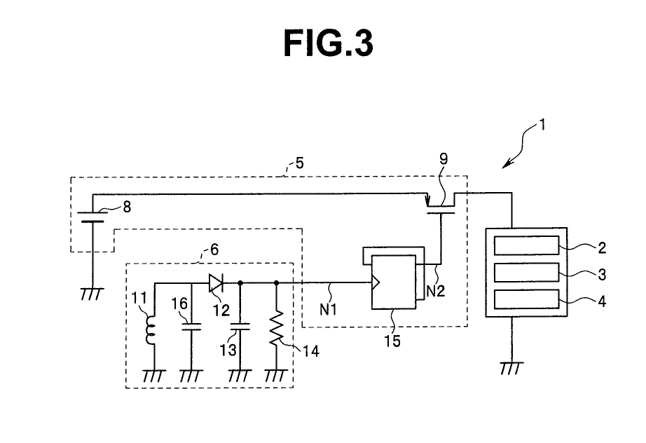

FIG. 1 shows an outline of the configuration of an in-vivo observation system showing the present embodiment; FIG. 2 shows an outline of the configuration of an electric circuit of an AC magnetic field generating apparatus that is included in a power source starter of FIG. 1; and FIG. 3 shows an outline of the configuration of an electric circuit of the capsule endoscope of FIG. 1.

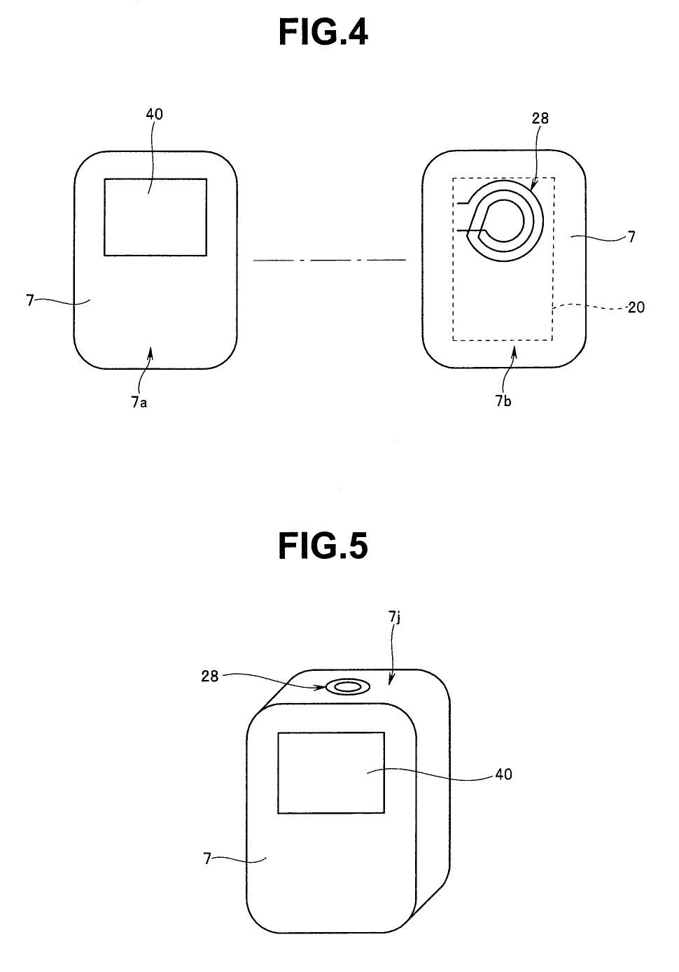

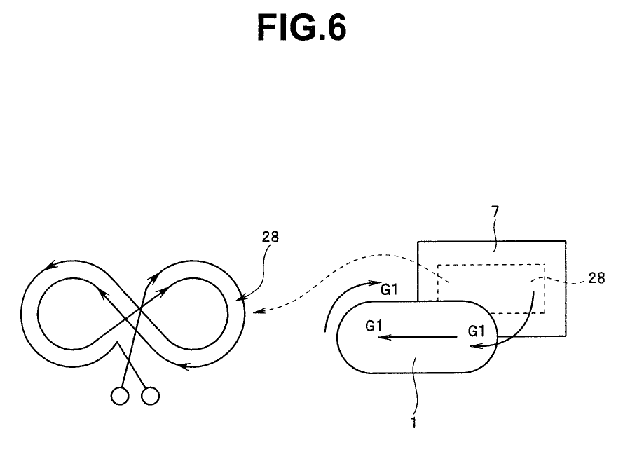

Moreover, FIG. 4 schematically shows an external appearance of the power source starter of FIG. 1; FIG. 5 shows a variant in which a primary side coil of a transmission antenna is provided on the top surface of the power source starter; and FIG. 6 shows a variant in which the planar shape of the primary side coil of the transmission antenna of FIG. 4 is formed into a figure of 8.

As shown in FIG. 1, a principal part of an in-vivo observation system 100 includes a capsule endoscope 1, and a power source starter 7 for applying an AC magnetic field G to the capsule endoscope 1, the power source starter 7 being disposed outside the capsule endoscope 1.

A principal part of the capsule endoscope 1 is configured to include: an illumination section 2 which is a living-body information acquiring section that acquires living-body information in a living body; an image pickup section 3 which is an living-body information acquiring section; a transmission section 4; a power supply control section 5; and a magnetic field detection section 6.

The illumination section 2 illuminates an observation site after the capsule endoscope 1 has come into an activated state, and the image pickup section 3 picks up images of the observation site after the capsule endoscope 1 has come into an activated state.

The transmission section 4 wirelessly transmits an image pickup signal, which is living-body information picked up by the image pickup section 3, to outside the living-body, for example, the power source starter 7, and the power supply control section 5 provides driving power to the illumination section 2, the image pickup section 3, and the transmission section 4.

The magnetic field detection section 6, a principal part of which includes: as shown in FIG. 3, a magnetic field detection coil 11; a half-wave rectification circuit made up of a diode 12 connected to the magnetic field detection coil 11 and a smoothing capacitor 13; a resonance capacitor 16 connected in parallel to the half-wave rectification circuit; and a resistor 14 connected in parallel to the smoothing capacitor 13, detects an AC magnetic field G that is generated from the outside, for example, the power source starter 7 and outputs the detection result as an electric signal to the power supply control section 5.

The power supply control section 5, a principal part of which includes: a cell 8 that constitutes a battery which is the power source section; a frequency dividing circuit 15 that performs a divide-by-2 operation of an electric signal (detection signal) from the magnetic detection section 6; and a P-channel type FET 9, the drain of which is connected to the cell 8, the gate of which is connected to the output of the frequency dividing circuit 15, and further the source of which is connected to the illumination section 2, the image pickup section 3, and the transmission section 4, controls the supply state of driving power supplied from the cell 8 to the illumination section 2, the image pickup section 3 and the transmission section 4, based on the inputted electric signal.

The power source starter 7, which generates an AC magnetic field G and receives an image pickup signal transmitted from the transmission section 4 of the capsule endoscope 1 by use of known means, has a flat outer surface having no protruded portion thereon to maintain the power source starter 7 in a clean state, as shown in FIG. 4. This is because if a protruded portion is formed on the outer surface, dusts and dirt etc. tend to remain around the protruded portion.

Moreover, the power source starter 7 is provided on the surface 7a thereof with a display section 40 on which an observed image that is picked up by the image pickup section 3 of the capsule endoscope 1 is displayed as a result of the power source starter 7 receiving an image pickup signal transmitted from the transmission section 4. That is, the display section 40 is integrally formed with the power source starter 7. It is noted that the display section 40 is made up of, for example, an LCD (Liquid Crystal Display) and an EL (Electro-Luminescence), etc.

Further, the power source starter 7 is provided with an AC magnetic field generating apparatus 20, which is a magnetic field generating section that generates an AC magnetic field G to perform the activation (On)/stopping (Off) of the capsule endoscope 1.

As shown in FIG. 2, a principal part of the AC magnetic field generating apparatus 20 is configured to include a power source 21 and an activation/stop signal generating section 30 that generates an AC magnetic field G which is an activation/stop signal for controlling the activation and stopping of the capsule endoscope 1.

A principal part of the activation/stop signal generating section 30 includes an oscillator 24, a timing generating circuit 25, a driver 26 for driving a transmission antenna 29, and the transmission antenna 29.

A principal part of the transmission antenna 29 includes a primary side coil 28 that generates an AC magnetic field, and a primary side capacitor 27. It is noted that the primary side coil 28 and the primary side capacitor 27 constitute a resonance circuit.

The transmission antenna 29, which transmits an AC magnetic field G which is an activation/stop signal to the capsule endoscope 1, is configured, as shown in FIG. 4, such that the primary side coil 28 formed into a loop-type planar antenna is provided in the back surface 7b of the power source starter 7.

It is noted that the primary side coil 28 may be provided, without being limited to the back surface 7b of the power source starter 7, in the top surface 7j of the power source starter 7 as shown in FIG. 5., and may be provided in anywhere within the range of the outer surface of the power source starter. Moreover, without being limited to the outer surface of the power source starter, the coil may be provided inside the power source starter.

Moreover, since the primary side coil 28 is formed as a planar antenna, it becomes easy to form the outer surface of the power source starter 7 into a flat surface having no protrusion as described above.

Further, as shown in FIG. 6, the planar shape of the primary side coil 28 may be a figure of 8. As a result of this, the orientation of the AC magnetic field G will be G1 as shown in FIG. 6, and it becomes easy to match the orientation of the magnetic field detection coil 11 of the capsule endoscope 1 to the orientation of the magnetic field G1, and the AC magnetic field G, which is an activation/stop signal transmitted from the primary side coil 28, can be stably transmitted to the capsule endoscope 1, thereby allowing the activation/stopping of the capsule endoscope 1 to be more reliably performed. It is noted that the planar shape of the primary side coil 28 may be any form without being limited to that of a loop-type or a figure of 8.

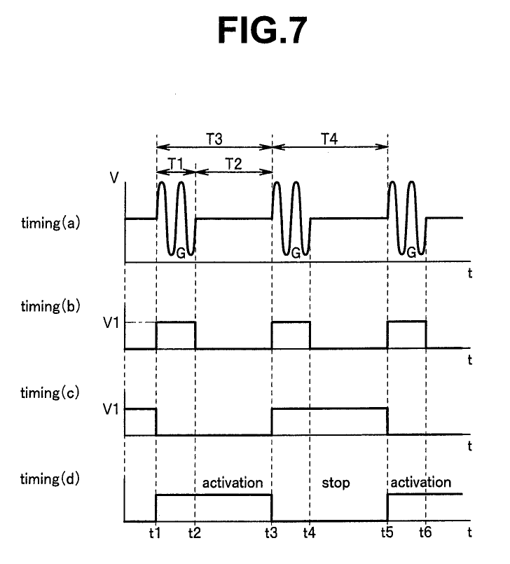

Next, the activation/stop operation of the capsule endoscope 1 by use of the AC magnetic field G will be described by using the above described FIGS. 1 to 3 and FIG. 7. FIG. 7 is a timing chart to show the activation and stop operations of the capsule endoscope by use of an AC magnetic field, in which timing (a) indicates a magnetic field generation state from the power source starter, timing (b) indicates a signal output of the magnetic field detection section of the capsule endoscope, timing (c) indicates a signal output of the frequency dividing circuit of the capsule endoscope, which is to be inputted to the gate of the P-channel type FET of the power supply control section, and timing (d) indicates a power supply state of the capsule endoscope.

As shown in the timing (a) of FIG. 7, first, when an AC magnetic field G is generated from the AC magnetic field generating apparatus 20 of the power source starter 7 at time t1, that is, an AC magnetic field G is transmitted from the transmission antenna 28 to the capsule endoscope 1, as shown in FIG. 3, an AC voltage is generated at both ends of the magnetic field detection coil 11 of the capsule endoscope 1 by electromagnetic induction, and is transformed into a DC voltage by a half-wave rectification circuit made up of a diode 12 and a smoothing capacitor 13, resulting in that the output potential (node N1) of the magnetic field detection section 6 becomes a high level (V1) as shown in the timing (b) of FIG. 7.

Next, as shown in the timing (a) of FIG. 7, when the generation of an AC magnetic field G from the power source starter 7 is stopped at time t2, that is, the transmission of an AC magnetic field G from the transmission antenna 28 to the capsule endoscope 1 is stopped, the output potential (node N1) of the magnetic field detection section 6 becomes a low level as shown in the timing (b) of FIG. 7.

Hereafter in the same fashion, the output of the magnetic field detection section 6 becomes a high level during a time period T1 in which an AC magnetic field G is being generated from the power source starter 7, and the output of the magnetic field detection section 6 during a time period T2 in which the AC magnetic field G is not being generated becomes a low level.

In the frequency dividing circuit 15 of the power supply control section 5, as shown in the timing (b) of FIG. 7 and the timing (c) of FIG. 7, the output potential (node N2) becomes a low level during a period from time t1 to time t3 (time period T3), and a high level during a period from time t3 to time t5 (time period T4) according to the output signal of the magnetic field detection section 6.

As a result, the P-channel type FET 9, the gate of which receives the output signal of the frequency dividing circuit 15, will be ON during a period from time t1 to time t3 (time period T3), and will be OFF during a period from time t3 to time t5 (time period T4). Therefore, as shown in the timing (d) of FIG. 7, power is supplied from the cell 8 to each circuit of the capsule endoscope 1 during the time period T3, and the supply of power will be stopped during the time period T4.

That is, every time when an AC magnetic field G is generated from the power source starter 7, power supply is started/stopped, and the capsule endoscope 1 is switched from an activated state to a stopped state, or from a stopped state to an activated state.

Therefore, the power source starter 7 becomes able to perform the switching control of the capsule endoscope 1 between an activated state and a stopped state. Thus, a generated AC magnetic field G has a kind of switching function.

Here, since the coil 11 in the magnetic field detection section 6 constitutes a resonance circuit with a resonance capacitor 16, matching the resonance frequency with the frequency of the AC magnetic field G generated from the power source starter 7 allows for stable control without erroneous activation or stopping of the capsule endoscope 1.

This is because, while the detection accuracy improves for the AC magnetic field G applied from the power source starter 7 thereby allowing for easy control of the activation and the stopping of the capsule endoscope 1, the detection accuracy declines for an unintended disturbance magnetic field, thereby inhibiting the activation and the stopping.

It is noted that although, in the configuration shown in FIG. 3, a half-wave rectification circuit is used as a smoothing circuit, it goes without saying that similar operation is possible, even if a full-wave rectification circuit is used. Further, although a P-channel type FET 9 is used as the switching means in the capsule endoscope 1, without being limited to this, other electronic switches may be used, provided that they have a similar function.

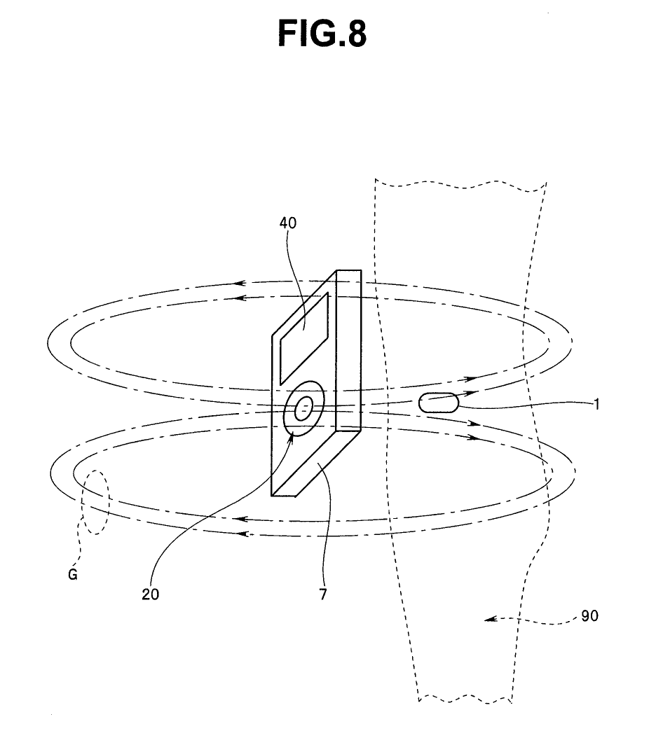

Next, the operation of the present embodiment will be described by using FIG. 8. FIG. 8 schematically shows a situation in which the activation/stopping of the capsule endoscope of FIG. 1, which has been introduced into a body after being passed through the mouth, is performed through the generation of an AC magnetic field by a power source starter, which includes a display section on which an observed image picked up by the capsule endoscope is displayed.

First, before the capsule endoscope 1 shown in FIG. 1 is passed through the mouth, an AC magnetic field G is generated from the AC magnetic field generating apparatus 20 of the power source starter 7 for a time period T1 to drive the capsule endoscope 1 by use of the above described means.

As a result, the image pickup section 3 of the capsule endoscope 1 starts image pickup and thereby an image pickup signal is transmitted from the transmission section 4 of the capsule endoscope 1 to the power source starter 7, and an observed image of the capsule endoscope 1 is displayed on the display section 40.

As a result of this, the operator can easily confirm that the capsule endoscope 1 normally gets activated, that is, the circuit that drives the illumination section 2, the circuit that drives the image pickup section 3, and the transmission section 4 normally get activated.

Thereafter, generating an AC magnetic field G from the power source starter 7 for a time period of T1 once again will result in that as described above, the capsule endoscope 1 comes into a stopped state. That is, the illumination section 2 and the image pickup section 3, etc. will stop.

In this stopped state, the capsule endoscope 1 is taken through the mouth by means such as swallowing by an examinee to be introduced into the body 90 of the examinee. Thereafter, upon elapse of a predetermined time, the power source starter 7 is moved close to the examinee as shown in FIG. 8, and the capsule endoscope 1 is brought into an activated state by generating an AC magnetic field G for a time period T1 by using the power source starter 7 once again.

As a result of that, when it can be judged that the capsule endoscope 1 has not reached the site to be observed from the observed image displayed on the display section 40, an AC magnetic field G is generated from the power source starter 7 for a time period T1 once again, to bring the capsule endoscope 7 into a stopped state. On the other hand, when it can be judged from the display section 40 that the capsule endoscope 1 has reached near the site to be observed, it is also possible to continue the observation of the site to be observed by using the display section 40.

In this way, it has been shown that in the present embodiment, even after the capsule endoscope 1 is passed through the mouth, the capsule endoscope 1 can be brought into an activated state or a stopped state every time when an AC magnetic field G is generated from the power source starter 7. Moreover, it has been shown that the display section 40 on which picked up images are displayed is integrated with the power source starter 7.

According to this configuration, since even after the capsule endoscope 1 has been passed through the mouth, the activation and stopping of the capsule endoscope 1 can be controlled every time when an AC magnetic field G is generated for a very short period of time from the power source starter 7, and the capsule endoscope 1 can be kept in a stopped state until it reaches the site to be observed; the energy dissipation of the cell 8 in the capsule endoscope 1 is prevented, and thereby an improvement in diagnostics capability can be expected.

Since it is possible to judge if the capsule endoscope 1 has reached the observation site while confirming the image inside the body 90 on the display section 40, even if a compact battery having a small battery capacity is incorporated in the capsule endoscope 1, it becomes possible to observe the site to be observed without concern for the battery capacity. As a result of this, since the capsule endoscope 1 can be down-sized for the part that the battery is down-sized, it is possible to provide a capsule endoscope 1 which can be swallowed with ease by the examinee.

Moreover, by bringing the capsule endoscope 1 into an activated state by using the power source starter 7 before the examinee takes in the capsule endoscope 1 through the mouth, the operator can easily judge that all of the circuit that drives the illumination section 2 of the capsule endoscope 1, the circuit that drives the image-pickup section 3, and the transmission section 4, etc. are operating, by confirming that an observed image picked up by the capsule endoscope 1 is being displayed on the display section 40.

As so far described, the power source of the capsule endoscope 1 after being passed through the mouth can be turned on/off only at a desired position from outside the body by use of a compact power source starter 7 which is integrated with the display section 40, so that it is possible to provide an in-vivo observation system 100 which can suppress useless power consumption of the battery of the capsule endoscope 1 after being passed through the mouth, and reduce the size of the battery, and a method for driving the in-vivo observation system 100.

It is noted that in the present embodiment, although the in-vivo observation apparatus has been described by taking an example of the capsule endoscope 1, of course, it is not limited to this and even when the in-vivo observation apparatus is applied to a medical capsule for pH measurement, a medical capsule for temperature measurement, and the like, similar effects as those of the present embodiment will be obtained.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An in-vivo observation system, comprising:
an in-vivo observation apparatus including
a living-body information acquiring section that acquires living-body information in a living body,
a transmission section that transmits the living-body information to outside the living body,
a power source section that supplies driving power to the living-body information acquiring section and the transmission section,
a magnetic field detection section that detects an AC magnetic field from outside and outputs a detection result as an electric signal, and
a power supply control section that controls the supply state of the driving power that is supplied from the power source section to the living-body information acquiring section and the transmission section, based on the electric signal; and
a power source starter for the in-vivo observation apparatus, which is disposed outside the in-vivo observation apparatus, and in which a magnetic field generating section that generates an AC magnetic field and a display section are integrally formed.

2. The in-vivo observation system according to claim 1, wherein
the power source starter receives the living-body information transmitted from the in-vivo observation apparatus, and
an observed image picked up by the in-vivo observation apparatus is displayed on the display section, the observed image being based on an image pickup signal acquired from the living-body information.

3. The in-vivo observation system according to claim 1, wherein the magnetic field generating section comprises an activation/stop signal generating section that generates a activation/stop signal for controlling the activation and stopping of the in-vivo observation apparatus.

4. The in-vivo observation system according to claim 2, wherein the magnetic field generating section comprises an activation/stop signal generating section that generates an activation/stop signal for controlling the activation and stopping of the in-vivo observation apparatus.

5. The in-vivo observation system according to claim 3, wherein the activation/stop signal generating section comprises: an oscillator; a transmission antenna that transmits the activation/stop signal to the in-vivo observation apparatus; and a driver that drives the transmission antenna.

6. The in-vivo observation system according to claim 4, wherein the activation/stop signal generating section comprises: an oscillator; a transmission antenna that transmits the activation/stop signal to the in-vivo observation apparatus; and a driver that drives the transmission antenna.

7. The in-vivo observation system according to claim 1, wherein the in-vivo observation apparatus is a capsule endoscope.

8. The in-vivo observation system according to claim 2, wherein the in-vivo observation apparatus is a capsule endoscope.

9. The in-vivo observation system according to claim 3, wherein the in-vivo observation apparatus is a capsule endoscope.

10. The in-vivo observation system according to claim 4, wherein the in-vivo observation apparatus is a capsule endoscope.

11. The in-vivo observation system according to claim 5, wherein the in-vivo observation apparatus is a capsule endoscope.

12. The in-vivo observation system according to claim 6, wherein the in-vivo observation apparatus is a capsule endoscope.

13. A method for driving an in-vivo observation system,
the in-vivo observation system comprising:
an in-vivo observation apparatus including
a living-body information acquiring section that acquires living-body information in a living body,
a transmission section that transmits the living-body information to outside the living body,
a power source section that supplies driving power to the living-body information acquiring section and the transmission section,
a magnetic field detection section that detects an AC magnetic field from outside and outputs a detection result as an electric signal, and
a power supply control section that controls the supply state of the driving power that is supplied from the power source section to the living-body information acquiring section and the transmission section, based on the electric signal; and
a power source starter for the in-vivo observation apparatus, which is disposed outside the in-vivo observation apparatus, and in which a magnetic field generating section that generates an AC magnetic field and a display section are integrally formed,
wherein the power supply state of the in-vivo observation apparatus is switched from On to Off, or Off to On every time when the AC magnetic field is emitted from the magnetic field generating section.

14. The method for driving the in-vivo observation system according to claim 13, wherein the in-vivo observation apparatus is a capsule endoscope.
